(19) United States
(12) Patent Application Publication
(10) Pub. No.: US 2016/0347688 A1
(43) Pub. Date: Dec. 1, 2016

(54) Olefin Production Process

(71) Applicant: LINDE AKTIENGESELLSCHAFT, München (DE)

(72) Inventors: Torben Höfel, München (DE); Clara Delhomme-Neudecker, München (DE); Ernst Haidegger, Riemerling (DE); Harald Schmaderer, Wolfratshausen (DE); Heinz Zimmermann, München (DE); Nicole Schödel, München (DE); Gunther Kracker-Semler, Unterhaching (DE)

(21) Appl. No.: 15/116,997

(22) PCT Filed: Feb. 6, 2015
(86) PCT No.: PCT/EP2015/052514

§ 371 Date: Aug. 5, 2016

(30) Foreign Application Priority Data
Feb. 6, 2014 (DE) 10 2014 001 652.8
Apr. 3, 2014 (EP) 14001241.0

(51) Int. Cl. C07C 7/00; C07C 7/163

(52) U.S. Cl. CPC C07C 7/005; C07C 7/163

(57) ABSTRACT

A method (100) for obtaining olefins is proposed, wherein a first gas mixture (b), which is produced by a steam cracking process (1), is at least partially used to form a first separation feedstock (f) which contains hydrocarbons with one to five carbon atoms, and from which at least a first separation product (g) and a second separation product (h, o) are produced, the first separation product (g) containing at least the greater proportion of the hydrocarbons with one carbon atom and of the hydrocarbons with two carbon atoms contained in the first separation feedstock (f), and the second separation product (h, o) containing at least the greater proportion of the hydrocarbons with four carbon atoms and of the hydrocarbons with five carbon atoms contained in the first separation feedstock (f), and wherein a second gas mixture (r) which is produced by an oxygenate-to-olefin process (2) is used at least partially to form a second separation feedstock (t) which contains hydrocarbons with one to five carbon atoms, and from which at least a third separation product (e, y) and a fourth separation product (l, z) are produced, the third separation product (e, y) containing at least the greater proportion of the hydrocarbons with one carbon atom and of the hydrocarbons with two carbon atoms contained in the second separation feedstock (t), and the fourth separation product (l, z) containing at least the greater proportion of the hydrocarbons with four or five carbon atoms contained in the second separation feedstock (t). The third separation product (e, y) is also at least partially used to form the first separation feedstock (f), and a third separation feedstock (h, l) (o, z) is formed from at least part of the fourth separation product (l, z) and of the second separation product (h, o) and is subjected to a separation (14).

The invention relates to an olefin production method according to the pre-characterizing clause of claim 1.

### PRIOR ART

Short-chain olefins such as ethylene and propylene can be produced by steam-cracking hydrocarbons, as explained in detail hereinafter. Alternative methods of obtaining short-chain olefins of this kind are the so-called oxygenate-to-olefin methods (in English: Oxygenates to Olefins, OTO).

By oxygenates are meant oxygen-containing compounds derived from saturated hydrocarbons, particularly ethers and alcohols. Oxygenates are used for example as fuel additives for increasing the octane number and as a lead substitute (cf. D. Barceló (ed.): Fuel Oxygenates, in D. Barceló and A. G. Kostianoy (ed.): The Handbook of Environmental Chemistry, vol. 5, Heidelberg: Springer, 2007). The addition of oxygenates to fuels ensures, among other things, clean burning in the engine and thereby reduces emissions.

Corresponding oxygenates are typically ethers and alcohols. Besides methyl tert. butyl ether (MTBE), it is also possible to use, for example, tert. amyl methyl ether (TAME), tert. amyl ethyl ether (TAEE), ethyl tert. butyl ether (ETBE) and diisopropyl ether (DIPE). Alcohols which may be used include for example methanol, ethanol and tert. butanol (TBA). The oxygenates also include, in particular, the dimethyl ether described hereinafter (DME, dimethyl ether). The invention is not limited to the fuel additives mentioned but is equally suitable for use with other oxygenates.

According to a common definition which is also used here, oxygenates are compounds which comprise at least one alkyl group covalently bonded to an oxygen atom. The at least one alkyl group may comprise up to five, up to four or up to three carbon atoms. In particular, the oxygenates which are of interest here comprise alkyl groups with one or two carbon atoms, particularly methyl groups. Preferably, they are monohydric alcohols and dialkyl ethers such as methanol and dimethyl ether or corresponding mixtures thereof.

In oxygenate-to-olefin methods, the oxygenates such as methanol or dimethyl ether are introduced into a reaction zone of a reactor in which a catalyst suitable for reacting the oxygenates has been provided. The catalyst typically contains a molecular sieve. Under the effect of the catalyst the oxygenates are reacted to form ethylene and propylene, for example. The catalysts and reaction conditions used in oxygenate-to-olefin methods are generally known to the skilled man.

Integrated methods and apparatus (combined apparatus) for producing hydrocarbons which comprise steam cracking processes and oxygenate-to-olefin processes or corresponding cracking furnaces and reactors are known and are described for example in WO 2011/057975 A2 or US 2013/0172627 A1.

Integrated methods of this kind are advantageous because typically not only the desired short-chain olefins are formed in the oxygenate-to-olefin processes. A substantial proportion of the oxygenates is converted into paraffins and C4plus olefins (for the designations see below). At the same time, in steam cracking, the entire furnace feed is not cracked into short-chain olefins. As yet unreacted paraffins may be present in the cracked gas of corresponding cracking furnaces. Moreover, C4plus olefins including diolefins such as butadiene are typically found here. The compounds obtained depend in both cases on the feeds and reaction conditions used.

In the methods proposed in WO 2011/057975 A2 and US 2013/0172627 A1 the cracked gas of a cracking furnace and the offstream from an oxygenate-to-olefin reactor are combined in a joint separating unit and fractionated. A C4 fraction may be subjected to a further steam cracking and/or oxygenate-to-olefin process, for example after hydrogenation or separation of butadiene. The C4 fraction may be separated into predominantly olefinic and predominantly paraffinic partial fractions.

The separation in a common separation unit does not always prove satisfactory, however, particularly when gas mixtures with significantly different compositions are obtained in a corresponding integrated process combining the oxygenate-to-olefin process and the steam cracking process and/or when an existing steam cracking apparatus is to be expanded by an additional part for carrying out an oxygenate-to-olefin process.

### DISCLOSURE OF THE INVENTION

Against this background the present invention proposes a method for producing olefins having the features of claim 1. Preferred embodiments are the subject of the dependent claims and the description that follows.

Before the explanation of the features and advantages of the present invention, their basis and the terminology used will be explained.

The abbreviations used within the scope of this application in the conventional manner for hydrocarbon mixtures or hydrocarbon fractions are based on the carbon number of the compounds that are predominantly or exclusively contained. Thus, a “C1 fraction” is a fraction which predominantly or exclusively contains methane (but by convention also contains hydrogen in some cases, and is then also called a “C1minus” fraction). A “C2 fraction” on the other hand predominantly or exclusively contains ethane, ethylene and/or acetylene. A “C3 fraction” predominantly contains propane, propylene, methyl acetylene and/or propadiene. A “C4 fraction” predominantly or exclusively contains butane, butene, butadiene and/or butyne, wherein the respective isomers may be present in different amounts depending on the source of the C4 fraction. The same also applies to a “C5 fraction” and the higher fractions. Several such fractions may also be combined in one process and/or under one heading. For example, a “C2plus fraction” predominantly or exclusively contains hydrocarbons with two and hydrocarbons with more than two carbon atoms and a “C2minus fraction” predominantly or exclusively contains hydrocarbons with one carbon atom and hydrocarbons with two carbon atoms.

Liquid and gaseous streams may, in the terminology used here, be rich in or poor in one or more components, “rich” indicating a content of at least 90%, 95%, 99%, 99.5%, 99.9%, 99.99% or 99.999% and “poor” indicating a content of at most 10%, 5%, 1%, 0.1%, 0.01% or 0.001% on a molar, weight or volume basis. Liquid and gaseous streams may also, in the terminology used here, be enriched or depleted in one or more components, these terms also applying to a corresponding content in a starting mixture from which the liquid or gaseous stream was obtained. The liquid or gaseous stream is “enriched” if it contains at least 1.1 times, 1.5 times, 2 times, 5 times, 10 times, 100 times or 1,000 times the amount, “depleted” if it contains at most 0.9 times, 0.5 times, 0.1 times, 0.01 times or 0.001 times the amount of a corresponding component, based on the starting mixture. A stream containing “predominantly” one or more components contains these one or more components to at least 50% or is rich in them, as defined above.

A liquid or gaseous stream is “derived” from another liquid or gaseous stream (which is also referred to as the starting stream) if it comprises at least some components that were present in the starting stream or obtained therefrom. A stream derived in this way may be obtained from the starting stream by separating off or deriving a partial stream or one or more components, concentrating or depleting one or more components, chemically or physically reacting one or more components, heating, cooling, pressurising and the like.

Methods and apparatus for steam cracking hydrocarbons are known and are described for example in the article “Ethylene” in Ullmann's Encyclopedia of Industrial Chemistry, online since 15 Apr. 2007, DOI 10.1002/14356007.a10_045.pub2.

Steam cracking processes are carried out on a commercial scale predominantly in tubular reactors in which the reaction tubes, the so-called coils, may be operated individually or in groups under identical or different cracking conditions. Reaction tubes or sets of reaction tubes operated under identical or comparable cracking conditions and possibly also tube reactors operated under uniform cracking conditions are each referred to hereinafter as “cracking furnaces”. A cracking furnace, in the terminology used here, is thus a construction unit used for steam cracking which exposes a furnace feed to identical or comparable cracking conditions. A steam cracking apparatus may comprise one or more cracking furnaces.

The present application uses the terms “pressure level” and “temperature level” to characterise pressures and temperatures, the intention being to indicate that corresponding pressures and temperatures in a corresponding apparatus do not have to be used in the form of precise pressure or temperature values in order to implement the inventive concept. However, such pressures and temperatures typically vary within certain ranges which are for example ±1%, 5%, 10%, 20% or even 50% either side of a mean value. Corresponding pressure levels and temperature levels may be located in disjointed ranges or in ranges that overlap. In particular, pressure levels will include unavoidable or expected pressure losses caused, for example, by the effects of cooling. The same is true of temperature levels. The pressure levels given in bar are absolute pressures.

For the design and specific configuration of distillation columns and absorption columns of the kind that may be used within the scope of the present application reference may be made to textbooks on the subject (cf. for example Sattler, K.: Thermische Trennverfahren: Grundlagen, Auslegung, Apparate, [Thermal separation methods: Principles, Design, Apparatus], 3^{rd} edition 2001, Weinheim, Wiley-VCH).

### Advantages of the Invention

The present invention further develops known methods for producing olefins in which gas mixtures which are produced by a steam cracking process and an oxygenate-to-olefin process are subjected as a separation feedstock to a joint separation process. As already mentioned, such methods are known for example from WO 2011/057975 A2 and/or US 2013/0172627 A1.

The present invention proposes a method for obtaining olefins in which a first gas mixture which is produced by a steam cracking method is at least partially used to form a first separation feedstock which contains hydrocarbons with one to five carbon atoms. The first separation feedstock may contain other hydrocarbons and other compounds, in addition to the hydrocarbons with one to five carbon atoms. It may also predominantly or exclusively contain the hydrocarbons with one to five carbon atoms. As already mentioned above, the “formation” of a first separation feedstock comprises not only the use of the first gas mixture as a whole as the first separation feedstock; a method according to the invention may also comprise the use of only part of such a gas mixture, optionally after purification and pre-treatment steps. As also explained in detail hereinafter, during the formation of the first separation feedstock, a part of the gas mixture which is produced by an oxygenate-to-olefin process is also used to form this first separation feedstock by being mixed with the first gas mixture or a part thereof. The formation of a separation feedstock may comprise combining the streams used for this purpose at an upstream location of, or in a separation unit.

The content of hydrocarbons in the first separation feedstock depends on the operating conditions of the steam cracking process used and particularly the hydrocarbon feeds which are subjected to the steam cracking process. If light hydrocarbons, i.e. gas mixtures with hydrocarbons having one to four carbon atoms, are used in a steam cracking process of this kind, the first separation feedstock naturally contains a smaller proportion of hydrocarbons with five or more carbon atoms. When heavier hydrocarbon feeds are used, on the other hand, larger proportions of longer-chained hydrocarbons with five or more carbon atoms are to be expected in the gas mixture produced by the steam cracking process. The invention makes it possible to process gas mixtures of all kinds.

Within the scope of the present invention, at least a first separation product and a second separation product are produced from the first separation feedstock which is produced as described hereinbefore. The first and the second separation product may be produced in a joint separation unit, but it is also possible to produce the first separation product in a first separation unit of a sequence of separation units and to produce the second separation product downstream of this first separation unit in a second, third, etc. separation unit. Thus, the first separation product and the second separation product may be produced at the same point or at different points in a corresponding separation sequence.

According to the invention, the first separation product contains at least the greater proportion of the hydrocarbons with one carbon atom and of the hydrocarbons with two carbon atoms contained in the first separation feedstock and the second separation product contains at least the greater proportion of the hydrocarbons with four carbon atoms and of the hydrocarbons with five carbon atoms contained in the first separation feedstock. As explained hereinafter, the first separation product is, in particular, the gaseous top stream of a deethanizer or a depropanizer, as known from the prior art. The second separation product, on the other hand, is obtained for example as the sump product of a corresponding deethanizer or depropanizer in the form of a liquid fraction.

According to the invention a second gas mixture which is produced by an oxygenate-to-olefin process is used at least partially to form a second separation feedstock. For the “formation” of the second separation feedstock reference may be made to the explanations given regarding the first gas mixture and the first separation feedstock. The second separation feedstock also contains hydrocarbons with one to five carbon atoms. The second separation feedstock may also contain, in addition to the hydrocarbons with one to five carbon atoms, other hydrocarbons and other compounds. It may also predominantly or exclusively contain the hydrocarbons with one to five carbon atoms. Depending on the configuration of the oxygenate-to-olefin process, its feedstocks, etc., the first separation feedstock may contain different amounts of hydrocarbons of different chain lengths. In particular, a separation feedstock of this kind may also still contain residual oxygenate which was not reacted in the oxygenate-to-olefin process. The method according to the invention may, however, also comprise separating off corresponding oxygenates from the second gas mixture before the formation of the second separation feedstock.

From the second separation feedstock, at least a third separation product and a fourth separation product are produced. The third separation product contains at least the greater proportion of the hydrocarbons with one carbon atom and of the hydrocarbons with two carbon atoms contained in the second separation feedstock and the fourth separation product contains at least the greater proportion of the hydrocarbons with four carbon atoms and of the hydrocarbons with five carbon atoms contained in the second separation feedstock. According to the invention, the formation of the third and fourth separation products takes place separately from the formation of the first and second separation products from the first separation feedstock. For this purpose, according to the invention, a second deethanizer or a second depropanizer is provided, for example, which forms the third separation product as its top product and the fourth separation product as its sump product.

The present invention is now characterised in that the third separation product is also at least partially used to form the first separation feedstock. In particular, the third separation product is totally combined with the first gas mixture obtained in the steam cracking process, which has optionally been worked up and purified, and is subjected to separation to obtain the first and second separation products. In addition, a third separation feedstock is formed from at least part of the fourth separation product and from at least part of the second separation product and also subjected to separation.

In other words, within the scope of the present invention, the third separation product, for example a C2minus or C3minus fraction which is formed from an offstream from an oxygenate-to-olefin process, is fed into a separation feedstock which is essentially formed from an offstream of a steam cracking process. A corresponding C3plus or C4plus fraction of the offstream from the oxygenate-to-olefin process, however, is combined with a fraction of comparable composition which is obtained from the first separation feedstock. Thus, initially, at least one light fraction (poor in C4plus hydrocarbons) and at least one heavy fraction (poor in C2minus hydrocarbons) is produced from the offstream from an oxygenate-to-olefin process before the different fractions are combined at a suitable point with fractions of an offstream from a steam cracking process.

In one embodiment of the present invention, the second separation product may contain at least the greater proportion of the hydrocarbons with three carbon atoms contained in the first separation feedstock. In this case, the fourth separation product contains at least the greater proportion of the hydrocarbons with three carbon atoms contained in the second separation feedstock. This means, in other words, that both the first separation feedstock and the second separation feedstock are initially processed in a deethanizer.

With regard to the gas mixture obtained from the oxygenate-to-olefin process this means, for example, that a gas mixture of this kind is initially subjected to quenching and optionally oxygenate removal. Then typically compression to a pressure of about 20 bar takes place, in the course of which the gas mixture liquefies. The condensates obtained are optionally dried and then fed into the deethanizer mentioned above. Light constituents of the gas mixture which do not go into the liquid state during the above-mentioned pressurisation, as well as a top product from the deethanizer, are further compressed together with the total gas mixture obtained in a steam cracker, i.e. by the steam cracking process, and then separated. The quantity of top product from the deethanizer depends on the catalyst used (see below). If, for example, ZSM-5 or a comparable material is used, it is a comparatively small amount. The “total gas mixture” from the steam cracker is optionally also pre-treated, for example dried, freed from condensates, etc., as mentioned previously. The significantly larger amount of C3plus hydrocarbons from the oxygenate-to-olefin process can be fed directly into a C3plus processing in a joint separation sequence.

The advantage that can be achieved by this is that, even where there are considerable differences in the C2minus/C3plus ratio in the gas mixture from the oxygenate-to-olefin process and the gas mixture from the steam cracking process, corresponding parts of the apparatus can be produced more easily and cheaply. A further advantage is the improved separation: if the gas mixture obtained from the oxygenate-to-olefin process contains carbon dioxide, this will go over into the C2minus fraction, i.e. the third separation product. The carbon dioxide removal can then take place jointly in this fraction and the entire gas mixture obtained from the steam cracking process, without the comparatively large amount of C3plus hydrocarbons from the oxygenate-to-olefin process having to be mixed with the gas mixture from the steam cracking process. The latter might lead to carbon dioxide dissolving into the condensates so that it cannot be removed separately.

In another variant of the method according to the invention, the third separation product may contain at least the greater proportion of the hydrocarbons with three carbon atoms which are contained in the second separation feedstock. In contrast to the alternative described previously, this therefore means that the gas mixture from the oxygenate-to-olefin process is initially processed in a depropanizer. As before, the gas mixture from the oxygenate-to-olefin process is first of all quenched, compressed and optionally dried, for example. The compression only has to be to a comparatively low level, for example to 10 to 15 bar. Then, corresponding separation is carried out in the depropanizer.

In certain cases it does not make technical sense to eliminate oxygenates such as dimethyl ether from the entire gas mixture which is obtained by the oxygenate-to-olefin process. This may be the case, for example, when the pressure is too low and/or when a two-phase stream is present. In this case the process according to the invention in the embodiment described opens up the possibility of eliminating dimethyl ether from the C3minus stream. Here, too, an energy advantage is obtained as not all the gas obtained from the oxygenate-to-olefin process has to be mixed with the gas from the steam cracking process, but only the C3minus fraction from the oxygenate-to-olefin process has to be mixed with the gas from the steam cracking process and the C4plus fraction from the oxygenate-to-olefin process is combined with the corresponding C4plus fraction from the steam cracking process.

The third separation feedstock can be subjected to different treatment steps within the scope of the present invention. The third separation feedstock is, as already explained, at least part of the fourth separation product and at least part of the second separation product. For example, such treatment may encompass hydrogenation, in which unwanted compounds, such as small amounts of butadiene, present in the third separation feedstock can be eliminated.

It should be emphasised again that within the scope of the present invention the first separation product and the second separation product are produced in a first separation unit and the third separation product and the fourth separation product are produced in a second separation unit which is structurally separate from the first separation unit. By “structurally separate” is meant that corresponding separation units are not charged with a common fluid stream which is formed from the first separation feedstock and the second separation feedstock. The separations are initially carried out separately from one another.

In particular, the first separation unit and the second separation unit advantageously comprise at least one distillation column, for example the deethanizer or depropanizer mentioned above.

Advantageously, the first separation product and the third separation product are each produced using a corresponding distillation column.

The method according to the invention in particular does not encompass the separation of butadiene from corresponding fractions before combining them to form the third separation feedstock. In other words, the first separation feedstock contains butadiene and this largely goes over into the second separation product which is then combined with the fourth separation product. This represents a major difference between the present invention and processes as known from WO 2014/005998 A1, for example. The objective there is to facilitate the extraction of butadiene from the offstream of a steam cracking process. Because of this objective the process proposed therein proves to be significantly less flexible that the one used within the scope of the present invention.

The present invention advantageously encompasses eliminating the butadiene from the third separation feedstock after the latter has been formed. This may be done for example using butadiene extraction and/or (thorough) hydrogenation, as explained hereinbefore. The residue remaining can be conditioned, separated into fractions and/or at least partially fed back into the steam cracking process as a recycle stream.

The invention may operate with various catalysts in the oxygenate-to-olefin process. For example, zeolites such as ZSM-5 or SAPO-34 or functionally comparable materials may be used. The present invention is particularly suitable when ZSM-5 or a comparable material is used, as comparatively large amounts of longer-chained (C3plus) hydrocarbons and comparatively small amounts of shorter-chained (C2minus) hydrocarbons are then formed. As already mentioned, the latter may be separated in a separate deethanizer and further processed together with the entire gas mixture obtained from the steam cracking process. However, the invention is also generally suitable for use with SAPO-34 or comparable materials with which shorter-chained (C2minus) hydrocarbons tend to be formed.

The invention can also be implemented in an apparatus for obtaining olefins, which comprises means that are designed to use a first gas mixture produced by a steam cracking process at least partially to form a first separation feedstock which contains hydrocarbons with one to five carbon atoms, as explained hereinbefore. These means, referred to here as first fluid processing means, are also designed to produce, from the first separation feedstock, at least a first separation product and a second separation product, the first separation product containing at least the greater proportion of the hydrocarbons with one carbon atom and of the hydrocarbons with two carbon atoms contained in the first separation feedstock, and the second separation product containing at least the greater proportion of the hydrocarbons with four carbon atoms and of the hydrocarbons with five carbon atoms contained in the first separation feedstock.

Such an apparatus further comprises means that are designed to use a second gas mixture produced by an oxygenate-to-olefin process at least partially to form a second separation feedstock which contains hydrocarbons with one to five carbon atoms, as explained hereinbefore. These means, referred to here as second fluid processing means, are also designed to produce, from the second separation feedstock, at least a third separation product and a fourth separation product, the third separation product containing at least the greater proportion of the hydrocarbons with one carbon atom and of the hydrocarbons with two carbon atoms contained in the second separation feedstock and the fourth separation product containing at least the greater proportion of the hydrocarbons with four carbon atoms and of the hydrocarbons with five carbon atoms contained in the second separation feedstock.

Especially, an apparatus of this kind is characterised by means which are designed to use the third separation product at least partially to form the first separation feedstock (referred to here as third fluid processing means), and by means which are designed to form a third separation feedstock from at least some of the fourth separation product and from at least some of the second separation product and to subject this third separation feedstock to a separation (referred to here as fourth fluid processing means).

An apparatus of this kind is designed to perform all the embodiments of a method as explained hereinbefore and comprises all the means that enable it to do so. The apparatus thus benefits from the features and advantages of the invention explained hereinbefore, to which reference is therefore expressly made.

In particular, the present invention is also suitable for providing a corresponding apparatus by revamping an existing apparatus which is set up only for carrying out a steam cracking process and subsequently separating the gas mixture obtained. An apparatus of this kind that is to be revamped thus comprises the first fluid processing means mentioned hereinbefore. The revamping may be carried out by the provision of means that are designed for carrying out an oxygenate-to-olefin process, for example at least one oxygenate-to-olefin reactor, and the provision and connecting up of at least the above-mentioned second fluid processing means and the above-mentioned third fluid processing means. The fourth fluid processing means mentioned may also be provided and connected up, but these may also be at least partially present already for processing a corresponding C4plus fraction from the steam cracking process. In this case it may be necessary to increase the volume of the fourth fluid processing means.

The revamping of an existing apparatus may be advantageous particularly in the cases described hereinafter, which predominantly start from the use of ZSM-5 as catalyst material. However, as already mentioned, the invention may also be carried out using other catalyst materials.

When (completely or partially) changing the feedstock(s) to be processed in the steam cracking process, for example when switching from naphtha to gaseous feeds such as ethane-containing shale gas, there is a proportionate reduction in the hydrocarbons with three or more carbon atoms in the gas mixture obtained by the steam cracking process. Such a switch may be desirable because corresponding gas mixtures can be obtained cheaply in the form of shale gas, for example.

However, the total throughput of an apparatus of this kind is also limited, when reducing hydrocarbons with three or more carbon atoms in the gas mixture obtained by the steam cracking process, by the dimensions of the separation units provided in a corresponding separation sequence for removing and separating the shorter-chained hydrocarbons, for example a so-called C2 splitter for separating ethane and ethylene from one another. The cracking furnaces used and particularly the separation units for removing and separating the longer-chained (C3plus) hydrocarbons, would not be needed in the capacity provided in such cases, i.e. they would be running below capacity. The available capacity of the cracking furnaces for processing longer-chained hydrocarbons is not fully utilised here either.

This below-capacity operation can be compensated by the provision of the means for carrying out an oxygenate-to-olefin process and by the provision and attachment of the second fluid processing means, the third fluid processing means and optionally the fourth fluid processing means (which may possibly already be present to some extent). For example, if a parallel oxygenate-to-olefin reactor with a separate water wash and compression and a subsequent C2/03 separation is provided, the separation part can be used for processing the C3plus hydrocarbons. Small amounts of a C2 fraction from the oxygenate-to-olefin process can be further processed in an existing compressor and in the existing separation unit. The C4plus fraction formed (predominantly from the oxygenate-to-olefin process) can make up the load in the available capacity of the cracking furnaces by recycling.

The invention can also be used when the capacity of an apparatus set up exclusively or predominantly for steam cracking naphtha is to be expanded without increasing the amount of naphtha processed by the steam cracking process or within the scope of a reduction in the amount of naphtha processed by the steam cracking process. Instead of using only naphtha as feedstock in the steam cracking process, a C4plus recycle from an oxygenate-to-olefin reactor expanded within the scope of the revamp can also be fed into the existing cracking furnaces and a corresponding amount of naphtha can be saved. Starting from a process structure as illustrated in FIG. 1, for example, the capacities in the process groups of the steam cracking process would not change substantially, apart from the C3plus part. The increase in capacity would take place as a result of the additional propylene from the oxygenate-to-olefin part. In this case, the C3plus part can be enlarged comparatively easily.

The invention may also offer particular advantages if a steam cracking process with a predominantly C2/C3 feedstock, i.e. a so-called gas cracker, is to be supplemented by an oxygenate-to-olefin process. In this case, the comparatively small amount of C2 from the oxygenate-to-olefin process can be co-processed in the existing components to carry out the steam cracking process. A new C3plus part which was not needed for the original steam cracking process because of the feedstocks used can process the C3plus hydrocarbons from the steam cracking process and the C3plus hydrocarbons from the oxygenate-to-olefin apparatus. The C4plus fraction could be supplied to new cracking furnaces, thus necessitating a redesign of the warm part of the steam cracking process. Overall, the gas feed quantity can be reduced slightly so that the capacities of the crude gas compressor and the cold part are sufficient for the C2minus hydrocarbons from the oxygenate-to-olefin process and the products from the gas furnaces and the C4plus furnaces.

All the modification measures described also have the consequence, as the result of the addition of an oxygenate-to-olefin reactor, that the propylene/ethylene ratio is relatively high at the apparatus limits. A conventional modification measure (e.g. converting the feed from naphtha to gas) would have the opposite effect: a reduction in the propylene/ethylene ratio or an exclusive production of ethylene. Against the background of an expected shortage of supply of propylene in the future, modification measures which increase the propylene/ethylene ratio appear attractive. The present invention allows such a modification to be made.

The invention is described in more detail with reference to the attached drawings which show preferred embodiments of the invention.

### BRIEF DESCRIPTION OF THE DRAWINGS

FIG. 1 shows a method according to one embodiment of the invention, in schematic representation.

FIG. 2 shows a method according to one embodiment of the invention, in schematic representation.

FIG. 3 shows a method according to one embodiment of the invention, in schematic representation.

FIG. 4 shows a method according to one embodiment of the invention, in schematic representation.

In the Figures, corresponding elements are indicated by identical reference numerals and are not explained repeatedly, in the interests of simplicity.

### EMBODIMENTS OF THE INVENTION

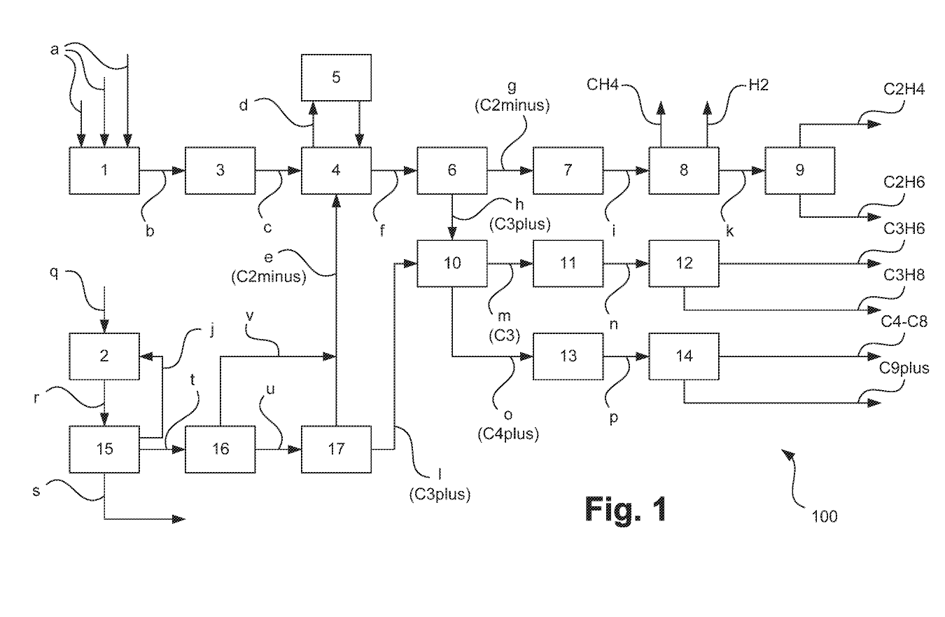

FIG. 1 schematically shows a method according to one embodiment of the invention in the form of a flow chart. The method as a whole is designated 100.

The method 100 comprises carrying out a steam cracking process 1 and an oxygenate-to-olefin process 2 in parallel. An apparatus in which the method 100 is implemented comprises corresponding means, i.e. at least one cracking furnace and at least one oxygenate-to-olefin reactor.

The steam cracking process 1 operates using one or more feed streams a which can be supplied to one or more cracking furnaces operated under the same or different cracking conditions, as explained hereinbefore. The streams a may comprise fresh feedstocks or any desired recycle streams from a corresponding method 100. Recycle streams may be, for example, ethane or propane streams and/or streams of hydrocarbons with four to eight carbon atoms (olefinic and paraffinic). Fresh feedstocks may, for example, be supplied in gaseous and/or liquid form, for example in the form of natural gas and/or naphtha.

Using the steam cracking process 1, a crude gas stream b is obtained which can be subjected to one or more preparation steps. In the embodiment shown, for example, an oil fractionation and/or a quenching are carried out in a process step 3. Process steam may be produced which can be recycled into the steam cracking process 1 (not shown).

A gas stream c obtained in step 3 is subjected for example to compression, pre-cooling and drying in a process step 4. Such a step 4 may also be supplemented by the elimination 5 of sour gas, with the formation of corresponding streams d (for example by diverting a gas stream between two compression stages from step 4 into the sour gas elimination 5 and feeding it back in again later). In the process step 4, a C2minus stream e may also be used at a pressure level of for example 20 bar (so-called medium-pressure C2minus stream) which is formed from a corresponding gas mixture of an oxygenate-to-olefin process 2, as explained hereinafter. The result of the joint use of the stream c and the C3minus stream e from the oxygenate-to-olefin process 2 is that a corresponding pre-treatment only has to be carried out once and does not have to be done again separately for the comparatively small amounts of C2minus hydrocarbons from an oxygenate-to-olefin process 2.

In the embodiment shown a stream f obtained from step 4, particularly one which has been compressed and partially liquefied and dried, is subjected, as a separation feedstock, to a deethanizer step 6 in which a C2minus fraction g at a pressure level of 35 bar, for example (high-pressure C2minus stream) and a C3plus fraction h is obtained. The further processing of the C3plus fraction h is explained hereinafter. The C2minus fraction g is subjected for example to a hydrogenation step 7 in which acetylene is hydrogenated to form ethylene, in particular.

A stream i further treated in this way is then subjected, for example, to a demethanizer step 8 in which methane CH4 and hydrogen H2 are separated off. A stream k thus freed from methane and hydrogen, which essentially still contains hydrocarbons with two carbon atoms, is subjected to a C2 separating step 9 (for example in a so-called C2 splitter) in which essentially ethylene C2H4 and ethane C2H6 are formed. The ethylene C2H4 is removed from the method 100 as a product, and the ethane C2H6 can be recycled into the steam cracking process 1, for example. If the process is designed accordingly, a method 100 according to the invention can also operate solely with recycled streams in the steam cracking process 1.

The C3plus stream h from the deethanizer step 6 is subjected to a depropanizer step 10. A C3plus fraction I, which is obtained from a gas mixture from the oxygenate-to-olefin process 2 as explained hereinafter, can be fed into the depropanizer step 10. This allows further joint processing of corresponding

C3plus fractions h and I. Thus, a common (third) separation feedstock is formed and is separated in the depropanizer step 10. As already mentioned, the formation of a separation feedstock may comprise combining streams used for this purpose at an upstream location or in a corresponding separation unit. The streams h and I may thus also be combined upstream of the depropanizer step 10 and subjected to the depropanizer step 10 as a combined stream. In the depropanizer step 10, a C3 fraction m is formed which can be worked up in one or more further process steps. For example, the C3 fraction m is subjected to a hydrogenation step 11 so that any methyl acetylene present as well as propadiene is reacted to form propylene. The stream thus processed, now designated n, is subjected for example to a C3 separating step 12 in which essentially propylene C3H6 and propane C3H8 are formed. The propylene C3H6, again, may be removed as product from a corresponding method 100, whereas the propane C3H8 may be recycled into the steam cracking process 1.

A C4plus fraction o also formed in the depropanizer step 10 is subjected for example to full or partial hydrogenation in a hydrogenation step 13. A stream p obtained is fed into a deoctanizer step 14 in which essentially a C4 to C8 stream and a C9plus stream (not referred to by abbreviations) are formed. The C9plus stream is removed from the process 100, whereas the C4 to C8 stream can be recycled back into the steam cracking process 1.

The recovery of the C2minus fraction and the C3plus fraction from the oxygenate-to-olefin process 2 will now be explained.

The oxygenate-to-olefin process 2 is particularly designed for reacting dimethyl ether, but methanol and other oxygenates, for example, may also be reacted. Corresponding oxygenates are supplied as stream q to one or more reactors and reacted to form a gas mixture r containing olefins. The gas mixture r, which contains at least or predominantly hydrocarbons with one to five carbon atoms, is subjected to an after-treatment step 15, for example water quenching and the elimination of oxygenates. Water obtained accordingly is drawn off as the stream s, and a stream t freed from oxygenates is fed into a step 16, which will be explained hereinafter. Any oxygenates recovered may be recycled as stream j into the oxygenate-to-olefin process 2.

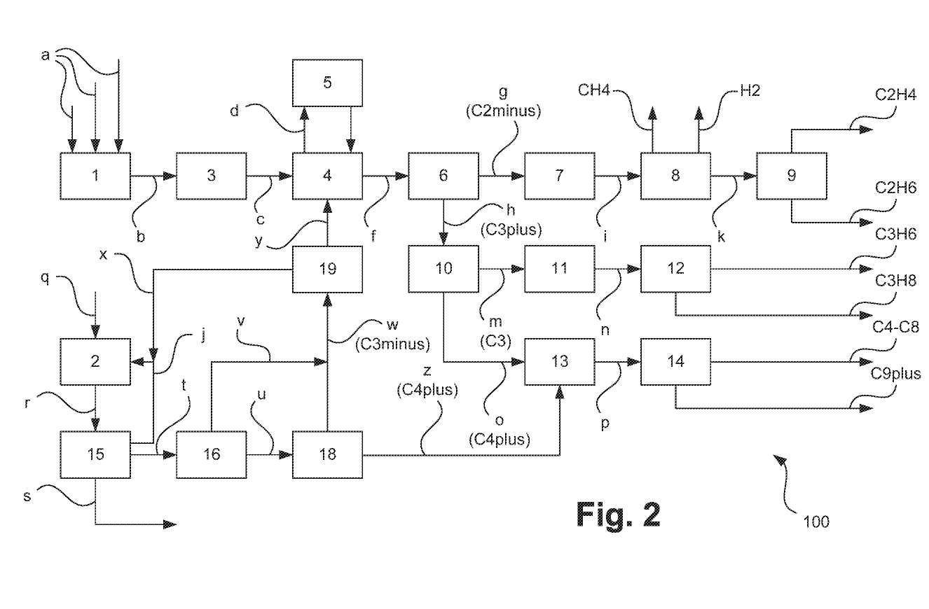

In step 16, which has already been mentioned, the stream t is compressed and optionally pre-cooled. As already explained above, condensable components of the stream t are condensed. Any condensate obtained is optionally dried and subjected as a liquid stream u to a deethanizer step 17 in which the above-mentioned C2minus fraction e and the C3plus fraction I are formed from the stream u. As already explained previously, these are fed into process step 4 or process step 10 (compression, pre-cooling and drying 4 on the one hand and depropanizer step 10, on the other hand). In the condensation step 16, non-condensable constituents of the stream t are combined as stream v with the C2minus stream e.

Whereas the embodiment of the method according to the invention shown in FIG. 1 operates using a deethanizer 17, the invention may be carried out in the same way using a depropanizer. This is shown in FIG. 2, which illustrates a corresponding variant of a method according to the invention in schematic form. The method is also generally designated 100. The steps 1 to 14 and the streams thus obtained do not differ substantially from those shown in FIG. 1. However, it should be pointed out that, in the embodiment shown in FIG. 2, a C3minus stream is supplied as stream y to the process step 4, and together with the C4plus stream o a C4plus stream z is fed into step 13, and a C3plus stream I is no longer fed into step 10. Thus, a joint (third) separation feedstock is formed from streams z and o and is separated in the depropanizer step 10. Here, too, the streams z and o may be combined upstream of the step 13 and subjected to step 13 as a combined stream.

Here, too, at least one oxygenate stream q is fed into the oxygenate-to-olefin process 2 and further processed as described hereinbefore, producing the streams r to v. The condensate in the form of stream u, however, is fed into a depropanizer step 18 in which a C3minus stream w is obtained. This is combined with the stream v and subjected to an oxygenate removal step 19. An oxygenate stream x separated off in the oxygenate removal step 19 is combined with the stream j and re-subjected to the oxygenate-to-olefin process 2. A C3minus stream y freed from oxygenates is then subjected to the process step 4 explained previously. A C4plus stream z obtained in the depropanizer step 18 is fed into the process step 13, as explained previously.

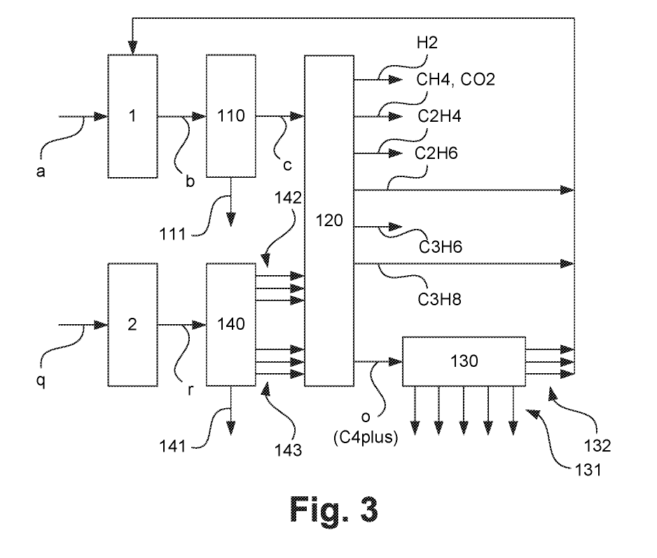

FIG. 3 illustrates a method according to the invention in generalised form. The elements used correspond to those described in connection with FIGS. 1 and 2, but in FIG. 3, in particular, a series of separation steps have been combined as blocks 110 to 140.

The feedstock streams a described previously are fed into the steam cracking process 1. A resulting stream b is subjected to the above-mentioned after-treatment steps 3 and 4 in FIGS. 1 and 2, which are shown here combined to form a block 110. In the combined after-treatment steps 110, for example, a water stream, oil and gasoline (indicated by arrow 111) are drawn off. The correspondingly cleaned gas mixture g is subjected to a joint separation step 120 in which the process steps 4 to 12 of FIGS. 1 and 2 are integrated, for example. In the joint separation step 120, as previously shown in detail with reference to FIGS. 1 and 2, hydrogen H2, methane CH4, carbon dioxide CO2, ethylene C2H4, ethane C2H6, propylene C3H6, propane C3H8 and C4plus hydrocarbons o are formed. Ethane C2H6 and propane C3H8 are recycled into the steam cracking process 1, as explained hereinbefore.

The C4plus stream o is subjected to a separation 130, explained in detail hereinbefore, which encompasses for example the process steps 13 and 14 shown in FIGS. 1 and 2, while products 131 can be removed and recycle fractions 132 are recycled into the steam cracking process 1.

The oxygenate-to-olefin process 2 operates using the oxygenate stream q mentioned previously. A stream r obtained is subjected to a pre-treatment, compression and preliminary separation 140, for example as described with reference to steps 15 to 18 and 15 to 19 of FIGS. 1 and 2. At least one stream 141 is formed which may contain water, oxygenates and gasoline, for example. Recycling is possible.

The method includes preliminary separation into one or more fractions 142 which are poor in C4plus hydrocarbons and one or more fractions 143 which are poor in C2minus hydrocarbons. Depending on the separation unit used (deethanizer and/or depropanizer) these fractions each contain C3 hydrocarbons (as explained in detail with reference to the streams e and I in FIG. 1 and the streams y and z in FIG. 2).

Revamping an existing apparatus in which the steam cracking process 1 and the processing and separation steps 110, 120 and 130 are already implemented includes the provision of components which implement the oxygenate-to-olefin process 2 and the subsequent separation 140.

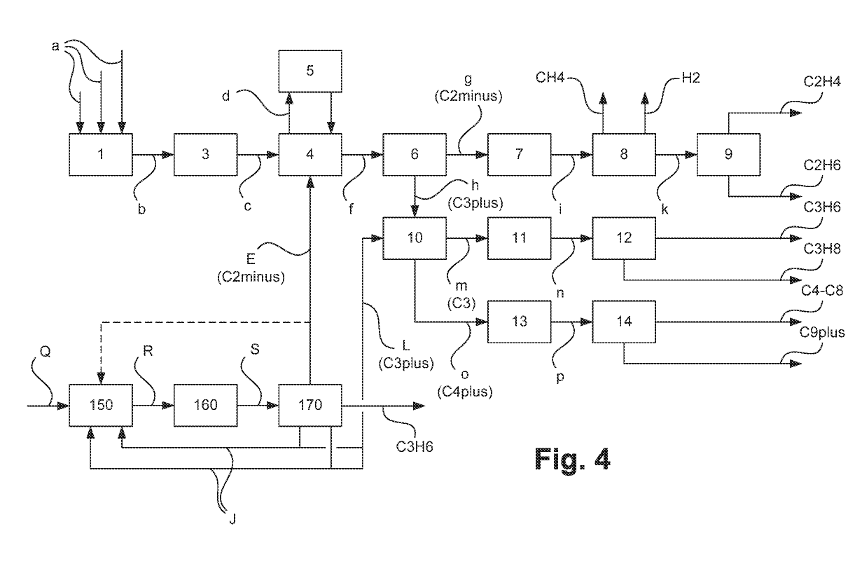

FIG. 4 shows a method according to another embodiment of the invention which results, in particular, from a subsequent expansion of an apparatus in which a steam cracking process 1 is implemented, to include corresponding steps of an oxygenate-to-olefin process. The steps 150 to 170 of the oxygenate-to-olefin process illustrated here correspond, for example, to the so-called Lurgi process. Apart from the differently performed steps 150 to 170, FIG. 4 essentially corresponds to FIG. 1, and reference is therefore made thereto regarding steps 1 to 14.

A methanol stream Q is fed into an oxygenate-to-olefin process, here designated 150. A gas mixture R is obtained which is subjected in particular to an after-treatment step such as water quenching, for example, and to compression and optionally drying 160. One or more compressed and dried, optionally partially liquefied streams S are subjected to a product separation 170. Streams such as liquid natural gas and gasoline, shown here as stream J, may be at least partially subjected to the oxygenate-to-olefin process 150 again, while another part is subjected to the depropanizer step 10 as the C3plus stream L.

In a product separation step 170, moreover, propylene C3H6 is formed, which can be extracted from a corresponding process.

Alternatively, the production of propylene C3H6 at this point may also be omitted by feeding a separate C3 fraction into the depropanizer step 10. In this case the recycle streams J and the stream L would no longer contain any propane C3H8 (C4plus).

A C2minus stream E produced in the step 170 is fed into the process step 4 and no longer recycled into the oxygenate-to-olefin process 150, as indicated by the dotted arrow, or discharged from the apparatus (not specifically shown here).

1. Method (100) for obtaining olefins, wherein
a first gas mixture (b), which is produced by a steam cracking process (1), is at least partially used to form a first separation feedstock (f) which contains hydrocarbons with one to five carbon atoms, and from which at least a first separation product (g) and a second separation product (h, o) are produced, the first separation product (g) containing at least the greater proportion of the hydrocarbons with one carbon atom and of the hydrocarbons with two carbon atoms contained in the first separation feedstock (f) and the second separation product (h, o) containing at least the greater proportion of the hydrocarbons with four carbon atoms and of the hydrocarbons with five carbon atoms contained in the first separation feedstock (f), and
a second gas mixture (r) which is produced by an oxygenate-to-olefin process (2) is at least partially used to form a second separation feedstock (t) which contains hydrocarbons with one to five carbon atoms, and from which at least a third separation product (e, y) and a fourth separation product (l, z) are produced, the third separation product (e, y) containing at least the greater proportion of the hydrocarbons with one carbon atom and of the hydrocarbons with two carbon atoms contained in the second separation feedstock (t) and the fourth separation product (l, z) containing at least the greater proportion of the hydrocarbons with four carbon atoms and of the hydrocarbons with five carbon atoms contained in the second separation feedstock (t),
characterised in that
the third separation product (e, y) is also at least partially used to form the first separation feedstock (f), and further characterized in that a third separation feedstock (h, l) (o, z) is formed from at least part of the fourth separation product (l, z) and from at least part of the second separation product (h, o) and is subjected to separation (14).

2. Method (100) according to claim 1, wherein the second separation product (h) further contains at least the greater proportion of the hydrocarbons with three carbon atoms contained in the first separation feedstock (f), and wherein the fourth separation product (l) further contains at least the greater proportion of the hydrocarbons with three carbon atoms contained in the second separation feedstock (t).

3. Method (100) according to claim 1, wherein the third separation product (y) further contains at least the greater proportion of the hydrocarbons with three carbon atoms contained in the second separation feedstock (t).

4. Method (100) according to one of the preceding claims, wherein at least part of the third separation feedstock (h, l) (o, z) is subjected, before or after the separation (14), to a hydrogenation (13) in which diolefins and/or olefins are at least partially hydrogenated.

5. Method (100) according to one of the preceding claims, wherein at least part of the third separation feedstock (h, l), (o. z) or at least a fraction formed therefrom by the separation (14) is at least partly recycled into the steam cracking process (1).

6. Method (100) according to one of the preceding claims, wherein the first (g) and the second separation product (h, o) are produced in a first separation unit (120) and the third (e, y) and fourth separation product (l, z) are produced in a second separation unit (140) which is structurally separate from the first separation unit (120).

7. Method (100) according to claim 6, wherein the first separation unit (120) and the second separation unit (140) each comprise at least one distillation column.

8. Method according to one of the preceding claims, wherein at least one deethanizer is used in the second separation unit (140).

9. Method according to one of the preceding claims, wherein at least one depropanizer is used in the second separation unit (140).

10. Method according to one of the preceding claims, wherein the first separation feedstock (f) contains butadiene and wherein the second separation product (h, o) contains the greater proportion of this butadiene.

11. Method according to claim 10, wherein after the formation of the third separation feedstock (h, l) (o, z) the butadiene is eliminated therefrom.

12. Method according to one of the preceding claims, wherein in the oxygenate-to-olefin process ZSM-5 or a material with a comparable product spectrum is used as catalyst material.
